(19) United States
(12) Patent Application Publication
(10) Pub. No.: US 2007/0060951 A1
(43) Pub. Date: Mar. 15, 2007

(54) Atrial tissue fixation device

(76) Inventor: Francis L. Shannon, Birmingham, MI (US)

Correspondence Address: BUTZEL LONG, STONERIDGE WEST, 41000 WOODWARD AVENUE, BLOOMFIELD HILLS, MI, 48304, US

(21) Appl. No.: 11/522,272

(22) Filed: Sep. 15, 2006

Related U.S. Application Data
(60) Provisional application No. 60/717,554, filed on Sep. 15, 2005.

(51) Int. Cl. A61B 17/08

(52) U.S. Cl. 606/216

(57) ABSTRACT

A simple device is provided that addresses the concerns of safety, efficiency and efficacy in closing the atrial appendage by conventional or minimally invasive cardiac surgical techniques. The device preferable is formed of a generally elliptical body with a through opening and at least two opposing magnetic serrations for closing the opening. During application, the opening is held open to prevent the magnetic serrations from prematurely coapting. The device is passed about the left atrial appendage through the opening to position the body about a neck of the left atrial appendage. Once the device is in place, the opposing magnetic serrations are permitted to coapt, closing the opening and occluding the orifice of the neck in a generally linear line of closure.

### BACKGROUND OF THE INVENTION

The present invention relates generally to a device and a method for the treatment of atrial fibrillation and its complications. Such a treatment is a large potential market for medical device companies. Current therapies have two primary goals: (1) blocking conduction of fibrillatory impulses around the atria by creating a series of electrical conduction blocks; and (2) isolating the left atrial appendage from the atrial blood pool to prevent clot formation and thromboembolism. There is controversy regarding the need to tie off or close the atrial appendage primarily because there is no simple device that safely and efficiently isolates the appendage from the atrial circulation.

Various methods and devices for closing the left atrial appendage are shown in the following patents:

U.S. Pat. No. 6,152,144 entitled “Method and Device for Left Atrial Appendage Occlusion”;

U.S. Pat. No. 6,488,689 entitled “Methods and Apparatus for Transpericardial Left Atrial Appendage Closure”;

U.S. Pat. No. 6,641,557 entitled “Method and Apparatus for Closing a Body Lumen”;

U.S. Pat. No. 6,652,555 entitled “Barrier Device for Covering the Ostium of Left Atrial Appendage”;

U.S. Pat. No. 6,666,861 entitled “Atrial Appendage Remodeling Device and Method”;

U.S. Pat. No. 6,712,804 entitled “Method of Closing an Opening in a Wall of the Heart”; and

U.S. Patent Application Publication No. US 2005/0149068.

These patents are incorporated by reference. Previous methods and devices do not provide effective long-term isolation of the left internal appendage (LAA) from the left atrium (LA). Specifically, there is a need to ensure that no cavity will persist between the LAA and the LA, allowing clot formation as has been found with the radial or “purse string” closure.

### SUMMARY OF THE INVENTION

The present invention concerns a proposal of a simple device to address the concerns of safety, efficiency and efficacy in closing the atrial appendage by conventional or minimally invasive cardiac surgical techniques. The device preferable is formed of a generally elliptical body with a through opening and at least two opposing magnetic serrations for closing the opening. During application, the opening is held open to prevent the magnetic serrations from prematurely coapting. The device is passed about the left atrial appendage through the opening to position the body about a neck of the left atrial appendage. Once the device is in place, the opposing magnetic serrations are permitted to coapt, closing the opening and occluding the orifice of the neck in a generally linear line of closure.

The body of the device is preferably formed of nitinol material and has an external surface area formed of a material, such as Dacron® for successful fibrous incorporation. The magnetic serrations are preferably graduated with maximal height serrations in the middle of the opening.

An advantage of using magnetic means for maintaining closure of the device about the LAA is the elimination of clamping the device closed, preventing uneven closure and/or tissue damage. The magnetic serrations of the present invention provide known and repeatable closure results of said opening about the left atrial appendage. This ensures linear closure and successful and continues isolation.

### DESCRIPTION OF THE DRAWINGS

The above, as well as other advantages of the present invention will become readily apparent to those skilled in the art from the following detailed description of a preferred embodiment when considered in the light of the accompanying drawings in which:

FIG. 1 is a perspective view of an atrial tissue fixation device in accordance with the present invention in a half open position;

FIG. 1a is a side elevation view of the device shown in FIG. 1;

FIG. 2 is front elevation view of the device shown in FIG. 1 in a fully open position;

FIG. 3 is a front elevation view of the device shown in FIG. 1 in a closed position;

FIG. 3a is a cross-sectional view taken along the line 3a-3a in FIG. 3;

FIG. 3b is a cross-sectional view taken along the line 3b-3b in FIG. 3;

FIG. 4 is a top plan view of the device shown in FIG. 1;

FIG. 5 is a cross-sectional view of a left atrial appendage;

FIGS. 6a-6b are schematic views of the common shapes of the left atrial appendage;

FIG. 7 is a view similar to FIG. 5 with the device shown in FIG. 1 installed;

FIG. 8 is a cross-sectional view taken along the line 8-8 in FIG. 7; and

FIG. 9 is a cross-sectional view of the left atrial appendage showing a prior art method of closure.

### DESCRIPTION OF THE PREFERRED EMBODIMENT

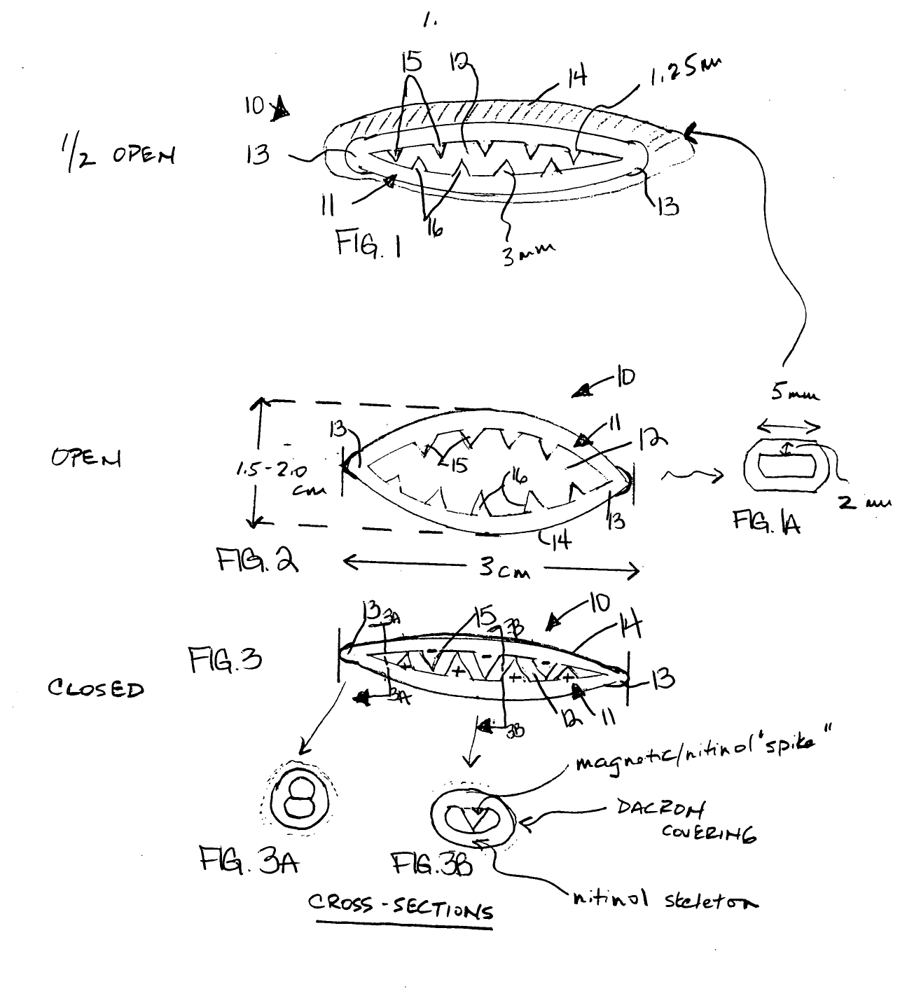

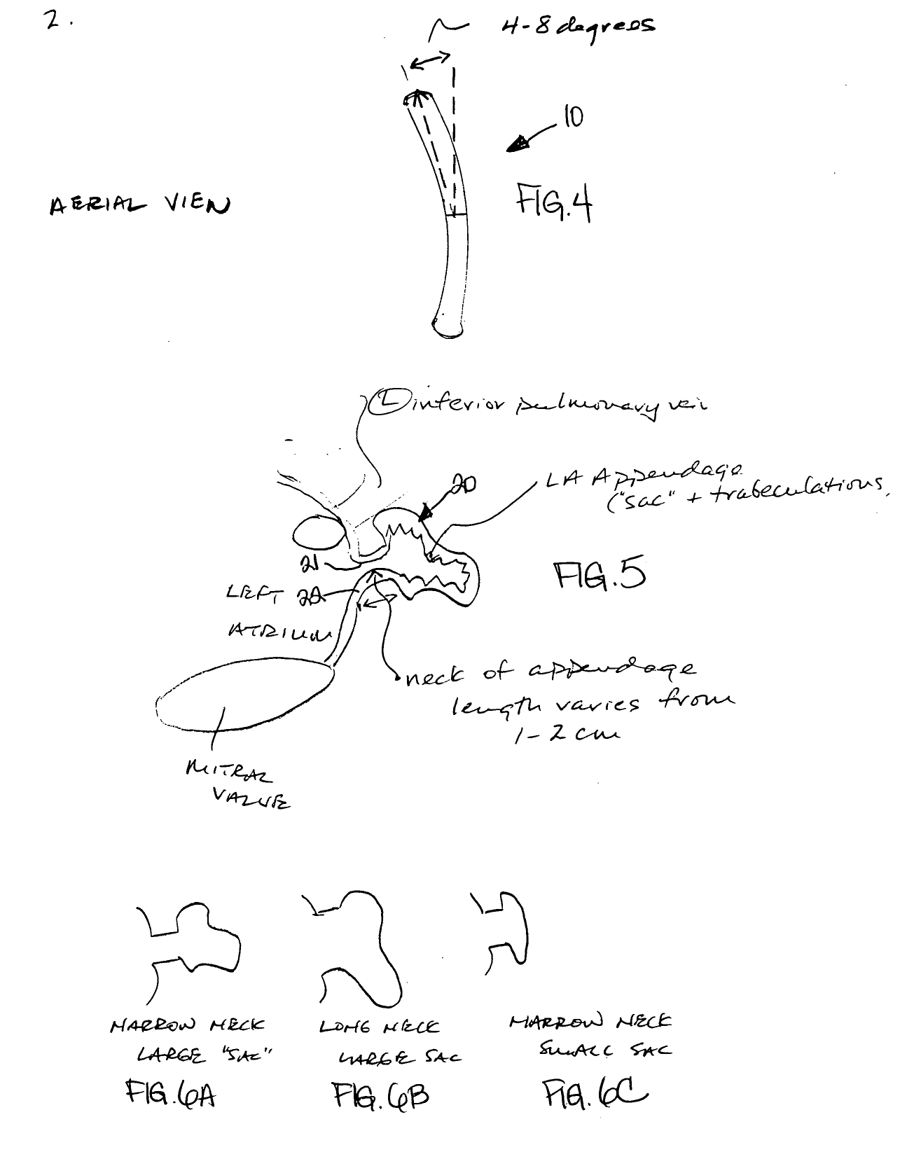

A magnetic atrial appendage fixation device 10 is shown in FIGS. 1-3 as having a body 11 with an elliptical cross section preferably made of nitinol material. The body 11 has a central through opening or cavity 12 and rounded ends 13. An external surface of the body 11 is preferably covered with a Dacron® cloth 14. An internal surface of the body 11 defining the opening 12 has a plurality of opposed magnetized spikes, teeth, or graduated serrations 15, 16 that apply sufficient force to permanently occlude the orifice 21 of the left atrial appendage 20 (see FIGS. 7 and 8). As shown in FIG. 3, the upper serrations 15 are of negative polarity and the lower serrations 16 are of positive polarity. However, the polarities can be reversed. Since nitinol material is nonferromagnetic, the serrations 15, 16 are formed of a ferromagnetic material and are attached to the body 11 by any suitable means such as bonding using medical grade epoxies and adhesives.

The magnetic atrial appendage occluder device 10 has a shape that resembles a hair “barrette” in that the upper and lower walls are flat and band-like with a width (front to rear) of approximately 5 mm and a thickness of approximately 2 mm (as shown in FIG. 1a). The ends 13 of the device 10 are rounded in the closed position (FIG. 3) to prevent erosion or piercing of nearby cardiac structures (e.g. left inferior pulmonary vein or circumflex coronary artery). In its closed state shown in FIGS. 3, 7 and 8, the device 10 closes the appendage orifice 21 flush with its origin from the left atrial wall 22, thereby creating a clean closure line that excludes the trabeculated wall of the appendage from the atrial circulation. Viewed internally, this line of closure is linear, rather than circular. Circular closure of the appendage by an external or internal purse string 30, as shown in FIG. 9, has the potential for post-closure thrombus formation with subsequent risk of thromboembolic migration after the appendage 20 is partially closed. Another device in development for left atrial appendage closure is of this circular type, with an animal model demonstrating progressive obliteration of the appendage over the first six post-operative weeks. Use of the external linear closure device 10 should eliminate the risks inherent in the circular appendage ligation or closure 30.

Use of magnetic force to effect closure of the device 10 has three advantages:
(1) The force applied is enough to close the mouth 21 of the appendage 20 without running the risk of avulsing the delicate atrial muscle, successfully addressing safety issues.
(2) The serrations 15, 16 guarantee fixation of the device 10 to the atrial tissue to effect initial sealing of the appendage 20 from the left atrial cavity and permit permanent closure by fibrous incorporation of the Dacron® coated barrette 10 into the atrial wall 22, successfully addressing efficacy issues.
(3) Magnetic force allows deployment of the device 10 in a simple way that makes the procedure efficient and amenable to both minimally invasive surgery, such as Videoscope-assisted thoracoscopic surgery via port access or robotic, or conventional open techniques such as median sternotomy. Further, the use of the magnetic force creates known and repeatable closure results, securing a stable closure without damaging nearby structure while maintaining isolation of the left atrial appendage with a linear rather than circular closure.

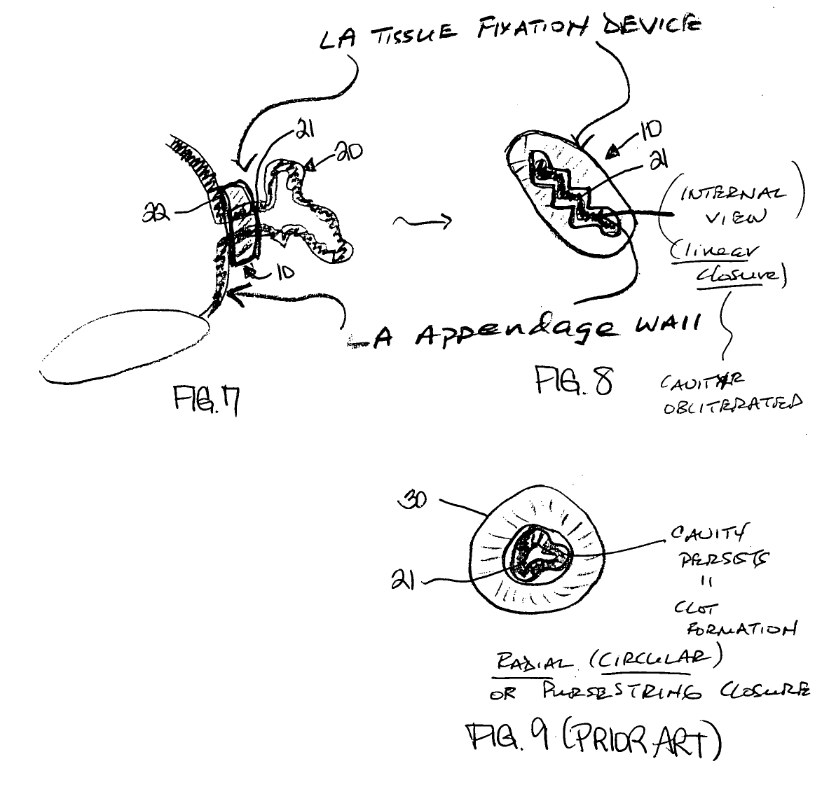

The length of the device 10 should accommodate the variable length of the closed appendage. Clinical experience indicates that the normal appendage has three configurations:
1. a narrow neck with a large trabeculated “sac” as shown in FIG. 6a;
2. a broad or long neck with a large trabeculated “sac” as shown in FIG. 6b; and
3. a narrow neck with a small “sac” as shown in FIG. 6c.

The barrette-shaped device 10 is ideal for closure of all of these configurations and the length of the walls (between the ends 13) of the body 11 should vary from approximately 2.0 cm to approximately 4.0 cm (3.0 cm being shown in FIG. 3). The remaining dimensions of the body 11 are preferably:

Width of the walls—approximately 5 mm;

Wall thickness—approximately 2 mm;

Dacron® fabric covering or external sleeve 14—approximately 0.5 to 1.0 mm thickness; and

Internal magnet serrations 15, 16—graduated with maximal height serrations in the middle of the opening 12 approximately 4 mm. The width at the base of each serration is approximately 2 mm and the center-to-center spacing is approximately 4 mm.

The serrations on the inferior surface should be polarized opposite of that on the superior surface.

When looked at from an “aerial” view (FIG. 4), the barrette-shaped device 10 should have a gentle curve so that it will accommodate the curve of the left atrial wall in this location. Measured from the center point of the device 10, this angle should be from approximately 4 degrees to approximately 8 degrees, with the greater curvature on longer (larger) ones of the device 10.

The deployment device for implanting the atrial tissue fixation device 10 should shield the magnets 15, 16 from each other so that they don't prematurely coapt. There should be a central opening in the deployment device that allows the atrial appendage 20 to be drawn through the device so that the fixation device 10 can be slipped onto the neck 21 of the atrial appendage. The deployment mechanism should be continuous so that the neck 21 of the atrium won't be avulsed from the atrial wall 30.

Since the device 10 is similar to staples and clips and requires an external surgical approach for implantation, it should not require more validation than the current endovascular atrial occluders.

In accordance with the provisions of the patent statutes, the present invention has been described in what is considered to represent its preferred embodiment. However, it should be noted that the invention can be practiced otherwise than as specifically illustrated and described without departing from its spirit or scope.

What is claimed is:

1. A device for permanently occluding the orifice of the left atrial appendage comprising:
a body having a generally elliptical cross section with a through opening; and
at least two opposing magnetized serrations on walls of said opening whereby when said body is maintained in an open position, the left atrial appendage can be passed through said opening to position said body about a neck of the left atrial appendage and when said body is allowed to close, said at least two opposing magnetized serrations attract, closing said opening in a generally linear line of closure.

2. The device according to claim 1 wherein said body is formed of nitinol material.

3. The device according to claim 1 wherein an external surface of said body is covered with a Dacron® material.

4. The device according to claim 1 wherein said serrations are graduated with maximal height serrations in the middle of said opening.

5. The device according to claim 1 wherein said external surface of said body is covered with a material that provides successful fibrous incorporation.

6. A method of permanently occluding the orifice of the left atrial appendage comprising the steps of:
a. providing a device having a generally elliptical body with a through opening and magnetic means for closing the opening;
b. holding the opening open;
c. passing the left atrial appendage through the opening to position the body about a neck of the left atrial appendage; and
d. permitting the magnetic means to close the opening thereby occluding the orifice of the neck in a generally linear line of closure.

7. The method according to claim 6, wherein said magnetic means comprises opposing magnetized serrations located on walls of said opening.

8. The method according to claim 7, wherein said serrations are graduated with maximal height serrations in the middle of said opening.

9. The method according to claim 6, wherein said magnetic means further provides known and repeatable closure results of said opening about the left atrial appendage.

10. The method according to claim 7 and further comprising the step of, after permitting said magnetic serrations to close said opening, permanently occluding the orifice of the left atrial appendage through magnetic attraction of opposing serrations.

11. The method according to claim 6 wherein said body is formed of nitinol material.

12. The method according to claim 6 and further comprising the step of providing said body with an external surface area covered with a material for successful fibrous incorporation.

13. The method according to claim 12 wherein said material is Dacron®.

14. A method of permanently occluding the orifice of the left atrial appendage comprising the steps of:
a. providing a device having a generally elliptical body with a through opening and at least two opposing magnetic serrations for closing the opening;
b. holding the opening open;
c. passing the left atrial appendage through the opening to position the body about a neck of the left atrial appendage; and
d. permitting said opposing magnetic serrations to close said opening thereby occluding the orifice of the neck in a generally linear line of closure.

15. The method according to claim 14, wherein said serrations are graduated with maximal height serrations in the middle of said opening.

16. The method according to claim 14, wherein said magnetic means further provide known and repeatable closure results of said opening about the left atrial appendage.

17. The method according to claim 15 and further comprising the step of, after permitting said magnetic serrations to close said opening, permanently occluding the orifice of the left atrial appendage through magnetic attraction of opposing serrations.

18. The method according to claim 14 wherein said body is formed of nitinol material.

19. The method according to claim 11 and further comprising the step of providing said body with an external surface area material for successful fibrous incorporation.

20. The method according to claim 19 wherein said material is Dacron®.
